(19) United States
(12) Patent Application Publication
(10) Pub. No.: US 2026/0256361 A1
(43) Pub. Date: Sep. 3, 2026

(54) SMART PET MONITORING

(71) Applicant: I.AM.SYMBOLIC, LLC, Los Angeles, CA (US)

(72) Inventor: William Adams, Los Angeles, CA (US)

(21) Appl. No.: 19/552,814

(22) Filed: Feb. 27, 2026

Related U.S. Application Data
(63) Continuation-in-part of application No. 30/038,944, filed on Dec. 19, 2025.
(60) Provisional application No. 63/765,337, filed on Feb. 28, 2025.
(60) Provisional application No. 63/945,030, filed on Dec. 19, 2025.

(51) Int. Cl. A61B 5/0205; A01K 15/02; A61B 5/00

(52) U.S. Cl. CPC A61B 5/0205; A01K 15/0201; A61B 5/0077; A61B 5/6802; A61B 5/7264; A61B 5/74; A61B 2503/40

(57) ABSTRACT

A wearable device for monitoring an animal is disclosed. The device includes a wearable article that supports an electronics module having one or more sensors and one or more processors. The sensors collect health data from the animal. The processors analyze the collected data to determine a health-related status, such as a physiological condition, behavior, or activity level. The resulting information can be sent to an external device or provided through outputs on the wearable device.

### CROSS-REFERENCE TO RELATED APPLICATIONS

This application claims the benefit of and priority to U.S. Provisional Patent Application Ser. No. 63/765,337, filed on Feb. 28, 2025, U.S. Provisional Patent Application Ser. No. 63/945,030, filed on Dec. 19, 2025, and to U.S. Design patent application Ser. No. 30/038,944, filed on Dec. 19, 2025, each of which are incorporated herein by reference in their entirety.

### BACKGROUND

Computers and computing systems have affected nearly every aspect of modern living. Computers are generally involved in work, recreation, healthcare, transportation, entertainment, household management, etc. One area of particular growth has been in the area of biometric monitoring. For example, many of the major technology companies around the world have released various forms of smart watches, smart bands, smart rings, or other devices that are able to gather various health readings from a wearer. These health readings may track exercise, steps, heart rate, oxygen levels, calories burned, and various other metrics. There is ongoing interest and need to expand these technologies and feature beyond their current forms.

The subject matter claimed herein is not limited to embodiments that solve any disadvantages or that operate only in environments such as those described above. Rather, this background is only provided to illustrate one exemplary technology area where at least one embodiment described herein may be practiced.

### BRIEF SUMMARY

Disclosed embodiments include a wearable device for smart monitoring of an animal. The wearable device may include a wearable article configured to be worn by the animal and an electronics module at least partially incorporated within the wearable article. The electronics module may include one or more sensors operatively connected to one or more processors. The one or more sensors may be configured to gather one or more health metrics from the animal. The electronics module may further include a non-transitory computer-readable storage medium storing instructions that, when executed by the one or more processors, cause the one or more processors to receive the health metrics and analyze the health metrics to generate a health-related output. The health-related output may be indicative of at least one of a physiological condition, behavioral state, or activity of the animal. The wearable device may further be configured to communicate the health-related output to an external computing device and/or to present the health-related output via one or more output components of the wearable device.

At least one additional or alternative embodiment may include a system for smart monitoring and interaction with an animal. The system may include a wearable device configured to be worn by the animal and an external computing device communicatively coupled to the wearable device. The wearable device may include one or more sensors and one or more processors configured to gather health metrics associated with the animal. The external computing device may include one or more processors and may cooperate with the wearable device to process data received from the wearable device.

The system may further include one or more non-transitory computer-readable media storing instructions that, when executed by the processors of the wearable device and/or the external computing device, cause the system to receive health metrics from the wearable device and analyze the health metrics using at least one artificial intelligence model. The artificial intelligence model may determine at least one of a physiological condition, behavioral state, or activity of the animal based on the received health metrics. The system may then generate an output associated with the animal based at least in part on the health metrics and the determined physiological condition, behavioral state, or activity. The output may be presented via the external computing device and/or through one or more output components of the wearable device.

At least one additional or alternative embodiment include a method for training a machine-learning model to classify animal behavior. The method may include collecting multi-point sensor data from multiple sensors of a wearable device positioned at different anatomical regions of an animal. The wearable device may generate a timecode signal, and video of the animal may be recorded using a camera configured to receive the timecode signal such that each video frame corresponds to a synchronized sensor-data interval.

The method may further include analyzing the recorded video to identify one or more activities of the animal and automatically labeling corresponding synchronized sensor-data intervals with the identified activities to produce labeled data segments. At least some of the labeled data segments may then be used to train the machine-learning model such that the trained model is configured to classify animal behavior based on sensor input alone.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered limiting in scope, embodiments will be described and explained with additional specificity and detail through the use of the accompanying drawings in which:

FIG. 1 shows a wearable device according to at least one embodiment described herein.

FIG. 2 shows an example electronics module according to at least one embodiment described herein.

FIG. 3 shows a flowchart representation of a method associated with a wearable device according to at least one embodiment described herein.

FIG. 4 illustrates a flowchart depicting a method for training an animal according to at least one embodiment described herein.

FIG. 5 shows a wearable device according to at least one embodiment described herein.

FIG. 6 illustrates a flowchart depicting a method of training, generating, and/or validating one or more machine-learning models according to at least one embodiment described herein.

FIG. 7 illustrates a system for smart monitoring of an animal according to at least one embodiment described herein.

FIG. 8 shows one example of an interaction between an owner and the wearable device according to at least one embodiment described herein

### DETAILED DESCRIPTION

Disclosed embodiments include a wearable device for smart monitoring of an animal. The device includes a wearable article that supports an electronics module having one or more sensors and one or more processors. The sensors collect health metrics from the animal. The processors analyze the collected data to determine a health-related status, such as a physiological condition, behavior, or activity level. The resulting information can be sent to an external device or provided through outputs on the wearable device.

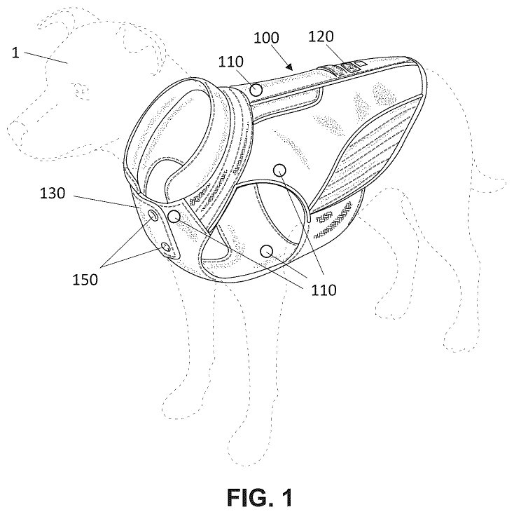

FIG. 1 shows a wearable device for smart animal monitoring according to at least one embodiment described herein. As shown in FIG. 1, the wearable device 100 may include an electronics module that includes one or more sensors 110 and one or more processors 120 to gather health metrics from an animal 1. The animal 1 may be a dog, cat, horse, and/or any other domesticated animal and may be a pet. While the one or more sensors 110 and one or more processors 120 are shown in FIG. 1 in a certain number and in certain positions, the present disclosure is not limited to any particular number of and/or positions for the one or more sensors 110 and the one or more processors 120. The one or more sensors 110 and the one or more processors 120 are thus shown as illustrative examples in FIG. 1.

In at least one embodiment, the wearable device 100 may comprise a wearable article 130 such as a collar, a shirt, a vest, a leg band, harness, and/or some other wearable article. The wearable article 130 may be made out of any suitable material, for example flexible textiles, woven fabrics, polymer-based materials, elastomeric materials, leather, mesh, neoprene, breathable synthetic blends, moisture-wicking fabrics, waterproof materials, reinforced composite materials, and/or any combination thereof. In at least one embodiment, the material may be selected to provide comfort to the animal while maintaining durability under outdoor, high-activity, or working conditions. The wearable article 130 may at least partially incorporate the electronics module and may provide protection to the components of the electronics module.

In at least one embodiment, the wearable article 130 may further include integrated channels, pockets, or structural supports configured to receive and retain the electronics module. In at least one embodiment, conductive threads, flexible printed circuits, and/or embedded wiring pathways may be integrated into the material to electrically couple the one or more sensors 110 positioned at different anatomical regions of the animal. The wearable article 130 may additionally include padding, adjustable straps, fastening mechanisms, and/or weight-distribution features configured to maintain proper sensor positioning while minimizing irritation or restriction of movement for the animal 1.

The wearable article 130 may include one or more pieces that are connected or integrally formed and may be sized and shaped to conform to the animal 1. The wearable article 130 may be manufactured using any suitable technique, for example additive manufacturing, injection molding, compression molding, thermoforming, sewing, weaving, knitting, lamination, overmolding, adhesive bonding, mechanical fastening, and/or other fabrication processes. In at least one embodiment, rigid structural portions and flexible textile portions may be combined to provide both support for the electronics module and comfort for the animal. The wearable article 130 may be formed as a single unitary structure or as multiple modular components that are detachable for cleaning, repair, or replacement.

The wearable device 100 may be configured to be worn by the animal 1. For example, the wearable device 100 may be configured to wrap around the animal 1 and affix to itself using an affixing component 140, which may in at least one embodiment include one or more buttons, hook and loop fasteners, snaps, and/or any other affixing component. As shown in FIG. 1, the wearable device 100 may provide sufficient space for the legs of the animal such that any movement of the animal 1 is not hindered by the wearable device 100.

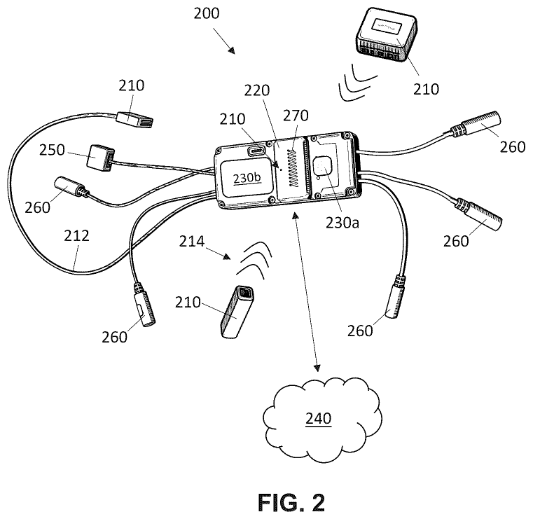

According to at least one embodiment, the electronics module (electronics module 200 of FIG. 2) may also include one or more light emitting diodes (LEDs), haptic sensors and/or actuators, and/or speakers. These may be configured to provide visual, physical, and/or auditory feedback and may be controlled by the one or more processors 120.

In at least one embodiment, the one or more sensors 110 may comprise one or more inertial measurement units (IMUs). The one or more IMUs may include 3-axis, 6-axis, and/or 9-axis gyroscopes, accelerometers, and/or magnetometers. Other non-limiting examples of the one or more sensors 110 may include pressure sensors, sound sensors, capacitive sensors, resistive sensors, temperature sensors, pulse-oximetry sensors, motion sensors, blood pressure sensors, blood oxygen sensors, doppler sensors, global positioning systems (GPS), microphones, skin-moisture sensors, and/or various other sensors. The one or more sensors 110 may be connected to the one or more processors 120 and may be configured to send data to the one or more processors 120. The data may correspond to a particular sensor. For example, an IMU may gather orientation, rotation, acceleration, heading data, and/or other state data in a 3D space. Other sensors such as the blood pressure sensor(s), blood oxygen sensor(s), temperature sensor(s), etc., may convey health data about the animal 1 and/or environmental data about the environment that surrounds the animal 1. The state data, health data, environmental data, and/or other data gathered by the one or more sensors 110 may be referred to collectively or individually herein as one or more health metrics.

The one or more sensors 110 may include one or more multi-point sensing arrays using a plurality of IMUs. In at least one embodiment, any number of IMUs may be used, for example, a number in a range from 2 to 9 IMUs may be used, distributed across different anatomical positions of the body of the animal 1. For example, IMUs may be positioned at the neck, chest, and dorsal regions of the wearable device 100 so that motion at each location can be independently measured and subsequently fused and/or combined to produce a higher-fidelity representation of the animal's posture, gait, and fine-grained behaviors. Such configurations may be contrasted with a single-sensor collar, which may provide only a coarse global motion signal. By capturing multi-axial accelerometer, gyroscope, and magnetometer readings at multiple anatomical anchor points, the wearable device 100 may enable advanced activity classification based on combining the spatially diverse measurements into a unified behavioral estimate. Such may be implemented by the one or more processors 120 and may enable the wearable device 100 to classify nuanced actions—such as sniffing, eating, toileting, or subtle head-turn patterns—that may not be reliably detected by a single IMU alone.

Additionally or alternatively, unlike single-sensor collar-based systems, at least one embodiment described herein may utilize spatially distributed sensor arrays, for example the one or more IMUs, that enable inter-sensor phase analysis and differential motion modeling. For example, differences in angular displacement between neck-mounted and dorsal-mounted IMUs may be used to detect subtle gait asymmetries indicative of early-stage musculoskeletal conditions. Such spatial-temporal modeling may not be achievable using a single inertial sensor.

In at least one embodiment, the one or more processors 120 may be configured to combine the data gathered from the accelerometers, gyroscopes, and/or magnetometers into a single representation. In at least one other embodiment, the one or more processors 120 may be configured to receive and combine multi-modal data from the other sensors 110 into a single representation. These multi-modal data may include health metrics from the temperature sensors, microphones, skin-moisture sensors, pressure sensors, vibration sensors, GPS receivers, and ultra-wideband (UWB) ranging modules. The aggregated data may be processed locally and/or streamed to an external computing device (external device 240 of FIG. 2) for time-series analysis, behavior modeling, and/or remote monitoring.

In at least one embodiment, the one or more processors 120 are configured to segment raw sensor signals into discrete time windows prior to analysis. For example, accelerometer and gyroscope data streams may be sampled at a rate between approximately 25 Hz and 400 Hz and partitioned into sliding windows having durations between approximately 100 milliseconds and 2 seconds, optionally with overlap between adjacent windows.

For each window, the one or more processors 120 may extract one or more features including, but not limited to: root-mean-square (RMS) acceleration, variance, skewness, kurtosis, dominant frequency components derived from fast Fourier transform (FFT) analysis, angular velocity magnitude, orientation change rate, cross-correlation between distributed IMUs, stride periodicity, and/or inter-sensor phase relationships.

In at least one embodiment, sensor fusion may be performed using a complementary filter, Kalman filter, extended Kalman filter, or other probabilistic state-estimation technique to combine multi-axis measurements from multiple anatomical locations into a unified posture and motion state vector. The resulting feature vectors may then be provided to one or more classification models for behavioral inference.

In at least one embodiment the wearable device 100 may be communicably connected with one or more processors (external device 240 of FIG. 2) that are external from the wearable device 100. The one or processors 120 and/or the external device 240 may be configured to receive and process the health metrics from the one or more sensors 110. For example, the one or more processors 120 may track the health metrics over time and map the health metrics to one or more indicators relating to the pet. For example, the one or more processors 120 may identify a change in health metrics that indicate the pet is moving less than normal, has a higher body temperature than normal, a different heart rate than normal, a different blood oxygen level than normal, or some other detectable change in health metrics. Based upon this change, the one or more processors 120 may generate an alert to an owner of the animal 1 that the animal 1 may be sick or may be facing some adverse condition.

In at least one embodiment, the one or more processors 120 can map the health metrics to particular health attributes or illness attributes. The particular health and/or illness attributes may be accessible to the one or more processors 120 in the form of one or more health metric templates. Each health metric template may include representative data that was gathered from a baseline healthy and/or ill animal against which the health metrics from the animal 1 may be compared by the one or more processors 120. The health metric template may be indicative of and/or correspond to a respiratory condition, cardiac condition, metabolic condition, or musculoskeletal condition.

For example, the one or more processors 120 may map the health metrics to a health metric template that indicates the animal 1 is working out. In at least one embodiment, the one or more processors 120 may map the health metrics to a health metric template that indicates the animal 1 has a particular type of illness, such as a respiratory infection.

In at least one embodiment, the health metric templates may include musculoskeletal-condition templates. For example, an arthritis template may include asymmetrical gait patterns detected by distributed IMUs, reduced stride length, increased time to transition between sitting and standing positions, reluctance to climb stairs as inferred from elevation and motion data, and/or increased rest intervals following minor exertion. Because the wearable device 100 may include multi-point IMU arrays, subtle differences in load distribution and joint motion may be detected earlier than through visual observation alone.

In at least one embodiment, each health metric template may include both population-level reference data and individualized baseline data for the specific animal 1. The individualized baseline may be generated during an initial calibration period in which the wearable device 100 collects health metrics while the animal 1 is presumed healthy. The one or more processors 120 may then weigh deviations from the individualized baseline more heavily than deviations from a generalized breed-level or species-level reference template. This dual-template approach may improve sensitivity and reduce false positives.

According to at least one embodiment, the health metric templates may be adaptive. For example, when a veterinarian confirms that the animal 1 has been diagnosed with a particular condition, the corresponding health metric template may be updated to reflect confirmed diagnostic parameters. The one or more processors 120 may then track progression or improvement relative to the diagnosed-condition template and may generate alerts when monitored metrics exceed predefined risk thresholds. Such alerts may be communicated to the external device 240 and displayed as trend graphs, severity scores, or recommended actions.

In at least one embodiment, each health metric template may be implemented as a structured data model comprising one or more statistical descriptors associated with a physiological or behavioral state. For example, a health metric template may include a mean feature vector u, a covariance matrix E, and one or more threshold values defining acceptable deviation ranges.

Deviations from a baseline health metric template may be calculated using a distance metric such as Euclidean distance, cosine similarity, Mahalanobis distance, dynamic time warping (DTW), or other time-series similarity measures. If a computed deviation exceeds a predetermined threshold, the one or more processors 120 may classify the current state as corresponding to a candidate condition represented by the template.

In at least one embodiment, the health metric templates may be adaptive and may be updated using incremental learning techniques, such as exponential moving averages, Bayesian updating, or periodic retraining using accumulated labeled data specific to the individual animal.

FIG. 2 shows an example electronics module according to at least one embodiment described herein. The electronics module 200 may be included with and/or disposed at least partially within the wearable device 100 and may include one or more rechargeable and/or disposable batteries. As shown in FIG. 2, the electronics module 200 may include one or more sensors 210 (which may be similar to or the same as the one or more sensors 110) and one or more processors 220 (which may be similar to or the same as the one or more processors 120). The electronics module 200 may also include one or more outputs such as light emitting diodes (LEDs) 250, haptic sensors and/or actuators 260, and/or speakers 270. The one or more sensors 210, the one or more processors 220, the one or more LEDs 250, the one or more haptic sensors and/or actuators 260, and one or more speakers 270 shown in FIG. 2 are not necessarily depicted to scale, rather; the components are shown as examples of certain types of components in the electronics module 200.

As described herein, the one or more sensors 210 may be connected to the one or more processors 220 such that data gathered by the one or more sensors 210 may be received, analyzed, and/or processed by the one or more processors 220. FIG. 2 shows that the one or more sensors 210 may be connected to the one or more processors 220 by a wired connection 212 and/or a wireless connection 214. The wired connection 212 may include one or more electrical conductors, signal traces, cables, buses, serial or parallel communication lines, or other physical transmission media configured to transmit power and/or data between the one or more sensors 210 and the one or more processors 220. In at least one embodiment, the wired connection 212 may include standardized communication interfaces such as I²C, SPI, UART, CAN, Ethernet, USB, or other suitable protocols.

The wireless connection 214 may comprise, for example, radio-frequency (RF) communication, Bluetooth®, Wi-Fi, Zigbee, cellular communication, near-field communication (NFC), infrared transmission, or other short-range or long-range wireless communication technologies. In at least one embodiment, the wireless connection 214 may include a transmitter and/or receiver associated with the one or more sensors 210 and/or the one or more processors 220.

FIG. 2 illustrates that the one or more sensors 210 may include different types of sensors and may be spaced apart from one another. This may allow the one or more sensors 210 to gather various different kinds of data as described herein from different anatomical positions relative to the wearable device 100 in which the electronics module 200 is incorporated.

FIG. 2 additionally illustrates that the one or more processors 220 may be communicably coupled with one or more external devices 240, for example using a wired and/or a wireless connection such as a Bluetooth, WiFi, ultra-wideband, 5G, and/or a cellular connection. The one or more external devices 240 may include a smartphone of the owner of the animal 1, a central server, and/or any other device wirelessly connected to the electronics module 200. The one or more processors 220 may be configured to forward the received health metrics from the one or more sensors 210 to the one or more external devices 240.

For example, when the external device 240 is a central server, the central server may have a larger amount of compute available relative to the one or more processors 220. Thus, any complex data analysis and/or processing may be performed by the external device 240 and then any outputs generated can be sent back to and received by the one or more processors 220. This may decrease a weight and allow for improved power efficiency of the electronics module 200.

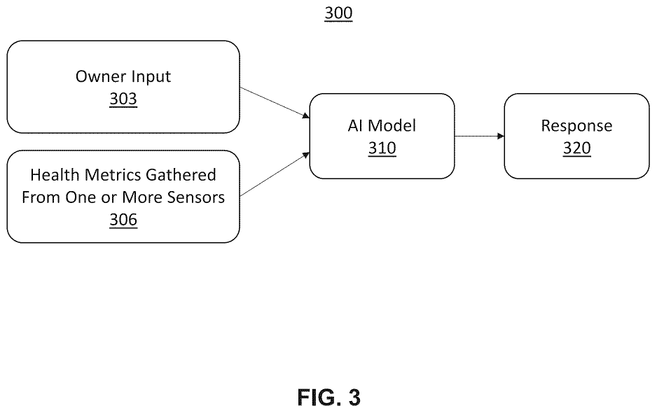

When the external device 240 is a smart phone of the owner of the animal 1, the external device 240 may be configured to send data to and receive one or more generated responses (as discussed with reference to FIG. 3) from the one or more processors 220. For example, the smartphone of the owner may connect to the one or more processors using a Bluetooth connection and may be used to provide one or more inputs to the one or more processors 220. The electronics module 200 may enable the owner to have the impression that the owner is communicating with the animal 1 as described herein.

FIG. 3 shows a flowchart representation of a method associated with a wearable device according to at least one embodiment described herein. The method 300 may be implemented at least partially by the wearable device 100, in particular at least partially by the one or more processors 120. As shown in FIG. 3, the one or more processors 120 may implement an artificial intelligence (AI) model 310 to track the health metrics and/or to interpret the health metrics. When using the wearable device 100, the owner may provide various inputs 303 to the AI model 310 that may include indications relating to the animal 1. Non limiting examples of the indications may include a type of the animal 1, a size of the animal 1, an age of the animal 1, and any other indication about a physical feature and/or characteristic of the animal 1. The AI model 310 may then use this information to map the health metrics to various categories and classes of outcomes.

In at least one embodiment, the AI model 310 may be local to and implemented by the one or more processors 120. In at least one embodiment, the AI model 310 may be implemented at least partially or fully remotely from the one or more processors 120 by the external device 240. For example, the AI model 310 may represent a computationally expensive component and may thus be performed by computer hardware that is remote to the wearable device 100. In at least one embodiment, the wearable device 100 may send the proper input data to the external device 240, which may perform analysis, processing, and response generation and then return the response 320 as an output to the wearable device 100.

In at least one embodiment, the AI model 310 may include one or more machine-learning architectures selected from convolutional neural networks (CNNs), recurrent neural networks (RNNs), long short-term memory networks (LSTMs), gated recurrent units (GRUs), temporal convolutional networks (TCNs), transformer-based time-series models, or hybrid architectures combining convolutional and recurrent layers.

For multi-point sensor array configurations, spatial-temporal feature maps derived from the IMUs may be processed using channel-wise convolution layers followed by temporal attention mechanisms configured to learn relationships between different anatomical regions of the animal.

The AI model 310 may be trained using supervised learning techniques in which labeled data segments are used to iteratively update model parameters through gradient-based optimization methods such as stochastic gradient descent (SGD), Adam optimization, RMSprop, or related techniques.

In at least one embodiment, the one or more processors 120 and/or the external device 240 may include a natural language processing module configured to allow an owner to communicate with his or her pet. For example, the AI model 310 may comprise one or more machine learning models, large language models (LLMs), generative pretrained transformer (GPT)-based models, retrieval-augmented generation (RAG) models, transformer-based sequence-to-sequence models, encoder-decoder architectures, reinforcement learning-enhanced language models, fine-tuned domain-specific language models, multimodal language models, and/or other natural language understanding and generation architectures configured to receive inputs and generate corresponding outputs.

In at least one embodiment, the AI model 310 may process text and/or speech from the owner and then generate and deliver a response 320 to the owner. As such, at least one embodiment may convey the impression that the animal 1 is replying to the owner, which may improve the awareness of the owner of any adverse or favorable conditions faced by the animal 1. Additionally, in at least one embodiment, the AI model 310 may utilize the health metrics received from the one or more sensors 110 to incorporate personality and/or mood into the responses. For example, if the animal 1 has an elevated heart rate and is active, the AI model 310 may incorporate an excited happy voice and/or tone into the response. Similarly, if the animal 1 has been laying down and/or sleeping for a period of time, the AI model 310 may incorporate a tired and quiet voice and/or tone into the response.

When the wearable device 100 includes at least a microphone and a speaker, the owner may ask questions verbally and receive synthesized audible responses generated by the AI model 310. As discussed in further detail herein, the AI model 310 may access both real-time health metrics and a historical database of prior observations to provide contextually-grounded responses that reflect the animal's recent and/or historical activities, emotional state, and environmental interactions. The AI model may in at least one embodiment be a RAG model that may use the historical activities, emotional state, and environmental interactions to refine the generated response of the AI model.

In at least one embodiment, the one or more processors 120 may receive a recording of the vocalizations of the animal 1 from the microphone(s). By way of example and not limitation, the vocalizations may include barks, brays, growls, meows, purrs, or any other vocalization form the animal 1. The AI model 310 may analyze the sound patterns and extract an emotional state of the animal from the recorded vocalizations. As such, the health metrics may be utilized by the one or more processors 120 to extrapolate and incorporate the animal's unique personality and/or apparent mood into the responses 320.

Additionally, in at least one embodiment, the AI model 310 may be configured to train or assist in training the animal 1. For example, the AI model 310 may track the animal 1 and issue commands to the animal 1. The AI model 310 may instruct the animal 1 to sit or lay down by outputting a vocal command using the one or more speakers 270 to the animal 1 based on receiving an input from the owner indicating that the AI model 310 should do so. The Al model 310 may also adaptively learn the animal's responses to the commands and general personality. This personality may also be used by the AI model 310 when generating the responses 320 to the owner's conversations with the AI model 310.

In at least one embodiment, the personality may be represented as one or more stored personality parameters, traits, or embeddings derived from the health metrics and/or owner inputs. For example, the AI model 310 may classify the animal 1 as energetic, cautious, food-motivated, anxious, independent, social, stubborn, compliant, or any combination of behavioral traits based on repeated observations of activity levels, response latency to commands, vocalization frequency, heart rate variability, and/or reinforcement success rates. These personality attributes may be quantified and stored as weighted variables within a personality profile associated with the animal 1. The personality profile may be used as an input into the AI model 310, for example by being incorporated into a prompt for the AI model 310.

In at least one embodiment, the personality profile associated with the animal 1 may be represented as a structured parameter set and/or embedding vector derived from aggregated behavioral statistics over time. For example, personality features may include normalized activity level scores, response latency metrics, reinforcement success rates, vocalization frequency, heart-rate variability trends, and/or behavioral persistence scores.

These values may be encoded into a numerical personality vector that is stored in association with the animal 1 and may be concatenated with one or more embeddings prior to submission to the AI model 310. In at least one embodiment, the personality vector may be used to adapt parameters of a language model, including temperature, response length constraints, vocabulary weighting, or tonal parameters in a text-to-speech synthesis module.

When generating the response 320, the AI model 310 may condition natural language outputs, tone, vocabulary selection, response length, enthusiasm level, and/or humor style on the stored personality profile. For example, if the animal 1 is classified as highly energetic and excitable, the generated response 320 may include higher-energy phrasing, shorter and more enthusiastic sentence structures, and/or excited vocal tones. Conversely, if the animal 1 is classified as calm or reserved, the generated response 320 may include slower-paced phrasing, softer tonal qualities, and more measured language. In at least one embodiment, the AI model 310 may modulate synthesized voice characteristics—including pitch, tempo, amplitude, and inflection—based on the personality profile and current health metrics.

Additionally, the AI model 310 may combine the learned personality parameters with real-time health metrics to generate contextually adaptive responses. For example, an energetic animal exhibiting reduced activity and elevated temperature may generate a response that reflects both its typically playful personality and its presently diminished condition. The presently diminished condition may include tiredness or discomfort in a manner consistent with learned character traits of the animal 1. In at least such a manner, the response 320 may reflect both stable personality characteristics and real-time physiological states extrapolated from the health metrics.

Additionally or alternatively, the wearable device 100 may utilize the health metrics to generate a long-term behavioral profile for each animal. Over time, the one or more processors 120 may identify deviations from the animal's typical activity patterns, eating behavior, or rest cycles, thereby assisting in early detection of potential illness or distress. Likewise, the AI model 310 may incorporate breed-specific traits, activity baselines, and personality characteristics to refine interpretations of the health metrics and to provide individualized recommendations related to care, exercise, or training routines.

In at least one embodiment, the method 300 may include generating the response 320 based on the health metrics received from the one or more sensors 110. Because the response 320 may be personalized to the animal 1 by being based on the health metrics gathered from the animal 1, the owner may be appraised of adverse conditions faced by the animal 1 more readily than if the owner were relying on visual observations alone.

In at least one embodiment, the method 300 may include receiving, at an AI model 310, owner input 303 and/or one or more health metrics 306, and generating, by the AI model 310, a response 320. The response 320 may include generated text and/or sounds. As discussed herein, the response 320 may be based on the owner input 303 and/or the health metrics 306 such that the response 320 indicates a current disposition and/or corresponds to a learned personality of the animal 1. The AI model 310 may thus give the impression that the owner is conversing with the animal 1 and may allow the owner a greater degree of knowledge about the health of the animal 1.

Additionally or alternatively, in at least one embodiment, the AI model 310 may include one or more machine-learning architectures selected from convolutional neural networks (CNNs), recurrent neural networks (RNNs), long short-term memory networks (LSTMs), gated recurrent units (GRUs), temporal convolutional networks (TCNs), transformer-based time-series models, or hybrid architectures combining convolutional and recurrent layers.

According to at least one embodiment, for multi-point sensor configurations, spatial-temporal feature maps derived from distributed IMUs may be processed using channel-wise convolution layers followed by temporal attention mechanisms configured to learn relationships between different anatomical regions of the animal.

In at least one additional or alternative embodiment, the AI model 310 may be trained using supervised learning techniques in which labeled data segments are used to iteratively update model parameters through gradient-based optimization methods such as stochastic gradient descent (SGD), Adam optimization, RMSprop, or related techniques.

In at least one embodiment, the one or more processors 120 and the AI model 310 may be configured to determine ideal times to exercise the animal 1, ideal times to groom or clean the animal 1, ideal times and amounts to feed the animal 1, suggest times for wellness checks, and/or various other similar activities. For example, the one or more processors 120 may identify the last time the animal 1 exercised based upon previously received health metrics 306. Based upon this information, the one or more processors 120 may suggest that the animal 1 should be exercised again. Further, the one or more processors 120 may also track the general wellbeing of the animal 1 and identify that the animal 1 needs more or less exercise relative to a different, similar animal. Thus, in at least one embodiment the response 320 may provide a personalized health plan to be developed for the animal 1, which may improve the overall health of the animal 1.

In at least one embodiment, the amount of needed exercise can be derived solely from the health metrics 306 from the animal 1 or may also be based upon various data input by the owner about and/or gathered from the animal 1, for example the species, breed, age, gender, and/or size of the animal 1.

In at least one embodiment, the owner of the animal 1, also referred to herein as a user, may be able to provide the owner input 303 to the wearable device 100 through one or more user interface elements 230 such as one or more touch sensitive buttons integrated into the wearable device 100. In at least one embodiment the one or more touch sensitive buttons include at least one TGO sensor. For example, the user may be able to power on or off these wearable device 100 through a power button 230a on the wearable device 100. Further, the user may be able to select various functions and/or provide various inputs (such as the owner input 303) through user interface elements 230 on the wearable device 100 such as touch sensitive buttons 230a, touch sensitive screens 230b, and/or other user interface elements 230 (see FIG. 2).

According to at least one embodiment, the animal 1 may be able to provide inputs to the wearable device 100 through pressure sensitive sensors, a microphone, TGO sensor, and/or some other sensor that the animal 1 can be trained to interact with. For example, the animal 1 may be a dog, and the dog may be trained to press against a particular portion of the wearable device 100 in order to activate a particular feature or functionality of the wearable device 100. The owner input 303 may thus include the input of the animal 1. In at least one embodiment, the owner input 303 and/or the health metrics 306 may be used by the one or more processors 120 to produce an output and/or the response 320 without the use of the Al model 310.

As described above with reference to FIG. 2, the wearable device 100 may provide one or more outputs using the one or more light emitting diodes (LEDs) 250, haptic actuators 260, and/or speakers 270. For example, the wearable device 100 may provide LED feedback, haptic feedback, and/or audible feedback in response to an input being provided from the user and/or the animal 1. For example, the one or more outputs may include a verbal command in the animal owner's voice. Additionally or alternatively, the one or more outputs may comprise a haptic vibration, a particular blinking of LEDs in the pathway of vision of the animal 1, or some other output. The one or more outputs may be generated by the one or more processors 120 based on the response 320. In at least one embodiment, the haptic vibration may include an LRA oscillation.

In at least one embodiment, the method 300 may include activating the one or more light emitting diodes (LEDs) 250, haptic actuators 260, and/or speakers 270 based on the response 320. For example, the response 320 and/or the one or more processors 120 may cause the wearable device 100 to output a pre-recorded vocal command from the owner using the speaker, and the method 300 may include outputting a pre-recorded vocal command and/or a generated voice clip based on the response 320. As another example, the response 320 may indicate that the wearable device 100 should illuminate the one or more LEDs, and the method 300 may include illuminating the one or more LEDs based on the response 320. As a further example, the response 320 and/or the one or more processors 120 may cause the haptic actuator 260 to vibrate, and the method 300 may include vibrating the haptic actuator based on the response 320 and/or using the one or more processors 120. In at least such a way, the coordinated activation of the one or more light emitting diodes (LEDs) 250, haptic actuators 260, and/or speakers 270 based on the response 320 may enable remote, real-time behavioral guidance and improved interaction with the animal 1, which may improve training consistency, responsiveness, and owner awareness.

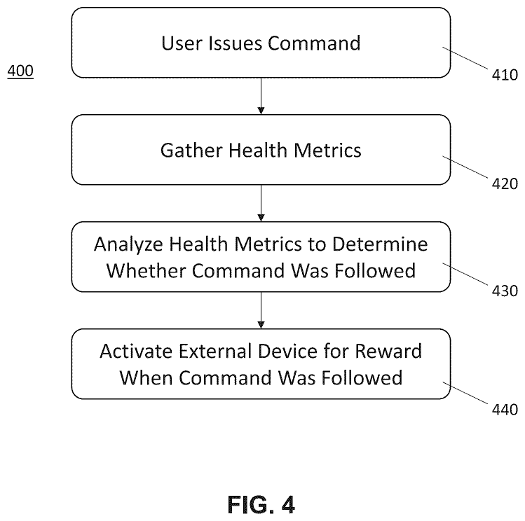

FIG. 4 illustrates a flowchart depicting a method for training an animal according to at least one embodiment described herein. The method 400 may be implemented at least partially by the wearable device 100. According to at least one embodiment, the user may record themselves saying several different commands using the microphone(s) of the wearable device 100 and/or the smartphone of the user. The wearable device 100 may then play those recorded commands to the animal 1, or the user may issue the command directly. In at least one embodiment, the wearable device 100 may gather health metrics from the one or more sensors 110 and then analyze the gathered health metrics to determine if the animal correctly followed the command. For example, the one or more sensors 110 may indicate whether the animal 1 lay down in response to a command to lie down.

The wearable device 100 may be configured to track a success of the animal 1 in following commands. For instance, the wearable device 100 may communicate the response to each command to an external device (such as external device 240 of FIG. 2). Further, in at least one embodiment, the wearable device 100 may be configured to activate an external device, such as a treat dispenser, in response to the animal 1 accomplishing a threshold number of correct responses to commands. The treat dispenser may be entirely external to or may be at least partially incorporated with the wearable device 100.

The method 400 may thus include a first step 410 of issuing a command, a second step 420 of gathering health metrics, a third step 430 of analyzing the health metrics to determine whether the command was followed, and an optional fourth step 440 of activating an external device for reward when the command was followed. The method 400 may provide for objective, sensor-based verification of compliance with a command rather than relying solely on human observation. By automatically detecting posture changes, motion signatures, orientation shifts, and/or physiological responses corresponding to the commanded behavior, the wearable device 100 may improve the training of the animal 1.

Additionally, the automated analysis and reward triggering may improve training consistency by delivering reinforcement with consistent timing relative to the detected behavior, which may strengthen associative learning in the animal 1. Because the wearable device 100 may log each issued command and corresponding compliance outcome, performance data may be generated, which may enable identification of trends, responsiveness improvements, areas requiring additional training, and/or behavior degradation over time. In at least one embodiment, such data may be communicated to the external device 240 for visualization, scoring, or remote trainer review.

Accordingly, at least one embodiment described herein may enhance training efficiency, enable remote or automated reinforcement, reduce training workload, and provide data-driven insight into the animal's learning progress and behavioral development.

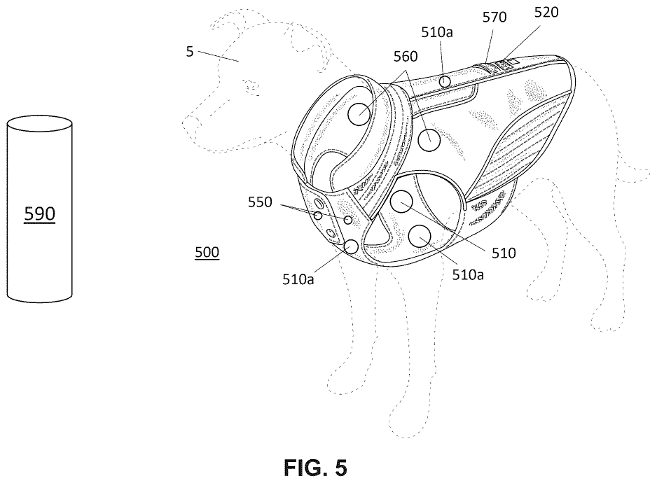

FIG. 5 shows a wearable device according to at least one embodiment described herein. FIG. 5 shows that the wearable device 500 may be similar to the wearable device 100 and may include one or more sensors 510 (which may be similar to or the same as the one or more sensors 110) and one or more processors 520 (which may be similar to or the same as the one or more processors 120). FIG. 5 also shows that the wearable device 500 may incorporate multiple transducers positioned on the wearable device 500 to enable two-way communication between the wearable device 500 and the animal 1. The transducers may include one or more LEDs 550, haptic actuators 560, and/or speakers 570, and/or other mechanisms capable of delivering discrete, non-verbal signals to the animal.

For example, the one or more haptic actuators 560 may deliver distinct vibration patterns associated with particular commands. In at least one embodiment, the one or more haptic actuators 560 may be controlled by the one or more processors 520 and/or as a result of the response of the Al model 310 to deliver the distinct vibration patterns. The wearable device 500 may thereby remotely instruct the animal to come, sit, turn left or right, or navigate toward a specified location. As one illustrative example, dogs can be trained to reliably associate specific haptic patterns with specific behaviors, enabling remote, silent interaction even when the owner is not co-located with the animal.

The transducers may be controlled by the one or more processors 520 based on received inputs and/or a response generated by an AI model (such as the AI model 310). For example, a user may remotely control the transducers by sending inputs to the one or more processors 520 to control a corresponding transducer. As another example, the response generated by the AI model may activate a corresponding transducer.

FIG. 5 also shows that the wearable device 500 may support autonomous reinforcement training routines for an animal 5. For example, where a remote treat dispenser 590 or other automated training aid is available, the wearable device 500 may integrate with external platforms to trigger rewards when the animal performs a desired action. In at least one embodiment, the wearable device 500 may use IFTTT-style automation services to integrate with external platforms.

In some implementations, the one or more processors 520 may process health metrics from the one or more sensors to determine whether the action was executed correctly—such as detecting that the animal 5 laid down, remained stationary for a threshold time, or navigated along an instructed path. Upon verifying the correct response, the wearable device 500 may automatically activate the remote treat dispenser 590 to reward the animal 5.

The one or more processors 520, similar to the one or more processors 120, may implement an AI model (AI model 310 of FIG. 3) and/or may be communicably connected to an external AI model (AI model 310 of FIG. 3). Such AI model(s) may incorporate trainer-defined behavioral rules, such as issuing a command no more than three times before pausing to prevent over-stimulation of the dog.

FIG. 5 also shows that when the animal 5 is a dog, the wearable device 500 may facilitate advanced working-dog functions, including scent-detection operations, search-and-rescue behavior, or medical-alert tasks. For example, a vest-mounted RFID reader may allow the dog to identify and report environmental markers or tagged objects. When the dog encounters a target scent or condition, the one or more processors 520 may detect an associated behavioral signature—such as a prolonged stationary posture or repeated directed sniffing—and may signal a handler of the dog by sending an alert to an external computing device operated by the handler and/or by modulating the dog's synthesized voice response generated by the wearable device 500. In some scenarios, the wearable device 500 may be used by service animals assisting individuals with medical needs; for example, the wearable device 500 may monitor the user's health-related reminders routed through a connected device and cue the animal 5 via the one or more haptic actuators 560 to approach the user and deliver an alert.

For example, the wearable device 500 may be communicatively coupled to a glucose monitoring system associated with the user, either directly or through a connected mobile device. When the glucose monitoring system detects that the user's blood glucose level has fallen below or risen above a predetermined threshold, the one or more processors 520 may receive a corresponding input. In response, the wearable device 500 may generate a predefined haptic cue, audio cue, or other signal directed to the animal 5, prompting the animal to approach the user, retrieve medication, activate an emergency alert system, and/or perform another trained assistance behavior.

In some configurations, the wearable device 500 may further monitor the response of the animal 5 using a multi-point, multi-modal sensor array to confirm whether the animal has initiated and/or completed the expected assistance task. For example, health metrics gathered from the one or more sensors may indicate that the animal transitioned from a resting posture and moved toward the user, remained proximate to the user for a threshold duration, and/or traveled to a designated medication storage location. Upon detecting successful completion of the task, the wearable device 500 may log the event and optionally transmit confirmation to an external computing device for recordkeeping or caregiver notification.

According to at least one embodiment, the wearable device 500 may be configured to train, assist, and improve performance of a working dog that performs scent-detection, tracking, mobility assistance, and/or other high-value tasks. In at least one embodiment, the wearable device 500 may include a multi-point array of distributed IMU sensors (such as the plurality of IMU sensors 510a) located at the neck, chest, and back of the harness that may be configured to continuously and/or discretely monitor the dog's posture, stride, and fine-scale movement signatures during a training routine. As the handler issues commands, for example verbally, through haptic cues, or through environmental stimuli, the wearable device 500 may gather health metrics from the one or more sensors 510 and may generate one or more corresponding sensor profiles for each respective sensor. Over time, these measurements may allow the wearable device 500 and the AI model(s) to build a precise library of motion patterns associated with successful and unsuccessful task execution. For instance, the multi-point IMU array can provide health metrics that allow the one or more processors 520 and/or the AI model(s) to distinguish between subtle differences in how a dog lowers its body when beginning a scent investigation versus the distinct stationary posture adopted when it has located a target and is awaiting further instructions.

In at least one embodiment when training a working dog for scent detection, the haptic actuators 560 may be used to provide remote directional cues. In one illustrative example, a pair of haptic actuators 560 mounted on the left and right shoulder regions of the wearable device 500 may deliver short vibration pulses to one or both of the haptic actuators 560 to instruct a dog to veer left or right while navigating indoor spaces such as warehouses, hotels, or airport cargo facilities. The multi-sensor array may provide the health metrics that enable the one or more processors 520 to confirm whether the dog executed the indicated turn by analyzing changes in angular velocity and direction from the array of IMUs. If the dog deviates from the intended path, the wearable device 500 may issue corrective haptic cues using the haptic actuators 560 while simultaneously logging the deviation for later review by the trainer/handler. At least one embodiment described herein may thus permit remote and silent guidance of the dog even when the handler is not physically nearby, which may be particularly valuable in constrained or hazardous environments.

In at least one embodiment, a dog may be trained to identify a particular scent. By way of example and not limitation, the particular scent may be a scent of contraband, explosives, bed bugs, invasive species, medical biomarkers, or any other scent the dog may be trained to identify. In at least one embodiment, the wearable device 500 may detect the characteristic behavior that occurs when the dog encounters the particular scent. For example, the dog may pause and adopt a distinctive stationary “alert” posture when it identifies a scent of interest. The IMUs, pressure sensors, and the one or more processors 520 may detect this behavior by recognizing a marked drop in translational motion combined with a predictable shift in body alignment. Upon detecting this signature, the wearable device 500 may interpret the behavior as a successful find and transmit an alert to the handler's device. In at least one embodiment, the handler need not follow the dog through every search area; instead, the dog may autonomously sweep a sequence of rooms or locations, and the wearable device 500 may notify the handler only when a target condition is met. This may allow the dog to perform the search more quickly and efficiently while allowing the handler to remotely supervise the dog.

The one or more LEDs 550, haptic actuators 560, and/or speakers 570 may also be used to reinforce learned detections and behaviors of the animal 5. For instance, after the dog has been conditioned to understand that a series of four short vibrations from the wearable device 500 corresponds to a “return to handler” command, the wearable device 500 may be configured to automatically issue such vibrations in response to a detection being logged by the one or more processors 520 based on the gathered health metrics. Health metrics confirming the dog's movement toward the handler may then trigger an automated reward through an external device such as the treat dispenser 590. In at least one embodiment, the AI model(s) implemented by and/or accessible to the wearable device 500 may dynamically modulate the timing and frequency of reinforcement. For example, the wearable device 500 may be configured to repeat a command no more than a predetermined number of times—such as three sequential cues—before pausing training to allow the dog to reset, which may prevent overstimulation and improving long-term learning consistency.

In at least one embodiment involving mobility or medical-alert service dogs, the one or more sensors 510 and the one or more LEDs 550, haptic actuators 560, and/or speakers 570 of the wearable device 500 may work together to improve safety and reliability when assisting an owner of the service dog. For example, the dog may be trained to respond to haptic cues that direct the dog to guide a visually impaired handler along a safe route. The wearable device 500 may interface with a navigation application of a device operated by the owner, converting turn-by-turn instructions into corresponding vibration patterns of the one or more haptic actuators 560 of the wearable device 500. For example, a long vibration on the left side of the harness may signal an upcoming left turn, while a rapid double-pulse on the chest region may instruct the dog to stop at a curb. As the dog moves, IMU measurements may be used by the one or more processors 520 to confirm orientation, step cadence, and/or whether the dog is encountering obstacles or irregularities in terrain. Deviations from expected movement patterns—such as abrupt halting that may indicate an obstruction—can be communicated back to the owner via audio feedback or displayed on the connected application.

FIG. 5 additionally shows that, during operation, the wearable device 500 may continue to collect high-resolution motion data that informs both real-time operation and long-term performance modeling. Over repeated sessions, the wearable device 500 may be configured to develop a behavior profile for the individual working dog, identifying the cadence of its typical search pattern, resting intervals, sniffing frequency, and other task-relevant metrics. If the dog begins to exhibit reduced activity, hesitation, or signs of fatigue, the wearable device 500 may flag these deviations as potential health or performance concerns, which may enable early intervention by the owner and reduce the risk of harm to the dog. Conversely, when the dog demonstrates highly reliable responses to certain haptic commands or excels in certain environmental conditions, the wearable device 500 may adapt its reinforcement strategies accordingly, progressively customizing the training flow to the dog's temperament, stamina, and job role.

In at least one embodiment, the wearable device 500 (and/or the wearable device 100), the external device 240, and the treat dispenser 590 may together form a system for smart monitoring and training of the animal 5. The external device 240 in the system may be communicatively coupled to the wearable device 500 and may include one or more second processors. The treat dispenser 590 in the system may be communicatively coupled to the wearable device 500 and/or the external device 240. The system may include one or more non-transitory computer-readable media storing instructions that, when executed by the one or more first processors and/or the one or more second processors, cause the system to: deliver a command to the animal; receive from the one or more sensors one or more health metrics associated with the animal; determine, based on the one or more health metrics, whether the animal performed a behavior corresponding to the command; and activate the treat dispenser to dispense a treat to the animal when the system determines that the animal performed the behavior corresponding to the command.

At least one embodiment described herein may thus demonstrate that a combination of multi-point sensing, targeted haptic actuation, and AI-driven interpretation may enable a robust and adaptive platform for training, guiding, and monitoring an animal, for example a working dog, across a wide range of operational environments. The wearable device 500 may in at least one embodiment not merely record activity; the wearable device 500 may form a closed feedback loop in which sensing informs haptic communication, haptic communication shapes behavior, and behavior in turn refines the sensing and classification models implemented by and/or accessible to the one or more processors. This may provide a continuously improving tool that enhances both the safety of the working dog and the effectiveness of the tasks the working dog performs.

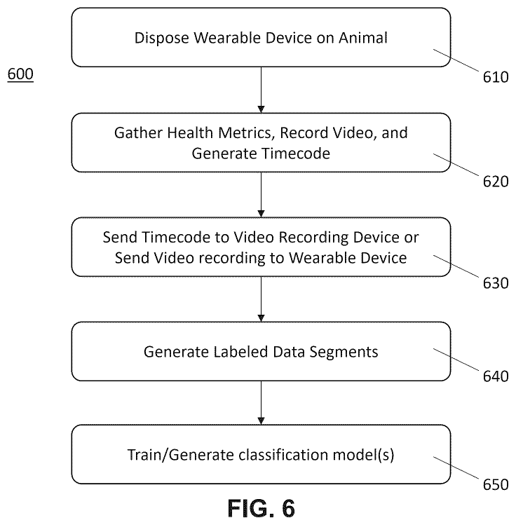

FIG. 6 illustrates a flowchart depicting a method of training, generating, and/or validating one or more machine-learning models according to at least one embodiment described herein. The method 600 may be implemented at least partially by the wearable device 100. In at least one embodiment, the wearable device 100 may thus also provide a comprehensive framework for training and validating one or more machine-learning models that operate on multi-sensor time-series data. The method 600 may include a first step 610 of disposing the wearable device 100 onto the animal while a handler wears a time-synchronized video recording device, such as a GoPro equipped with a dedicated time-code module. The wearable device 100 may operate as a master clock and may generate a precise timecode that is fed directly to the video recording device, which may produce video frames that align exactly with the data stream from the one or more sensors 110.

The method 600 may include a second step 620 of gathering health metrics from the one or more sensors 110, recording video using the video recording device, and generating a timecode. The method 600 may further include a third step 630 of sending the timecode to the video recording device, and/or sending the video recording to the wearable device 100, which may enable precise labeling of video frames that correspond in time to the gathered health metrics. This may provide one or more synchronized data segments. In at least one embodiment, a data segment includes at least some of the health metrics and one or more video recording frames that correspond in time to when the health metrics were gathered.

This alignment may support automated labeling pipelines in which a pre-trained AI model analyzes video frames to identify the ongoing activity and then assigns corresponding labels to the sensor-data segments. By way of example and not limitation, the ongoing activity may be identified as running, walking, eating, lying down, sniffing, toileting, or other activities. This approach may eliminate the need for labor-intensive manual annotation of time-series data and may improve the accuracy, scalability, and consistency of classification models that are trained using the labeled data.

The method 600 may include a fourth step 640 of generating labeled data segments. The wearable device 100 may generate the labeled data segments, or the wearable device 100 may transmit the video recording and the timecodes to an external device (such as external device 240), which may perform the labelling of the data segments. In at least one embodiment, the fourth step 640 may include a combination of manual labelling and computer-based labelling. In at least one embodiment, the computer-based labelling may include using an Al or machine learning model such as a vision-based classifier or an LLM configured to process video frames.

A labeled data segment may include a fixed amount of health metrics and a timecode, which may be generated based on the video recording at the corresponding time. As an illustrative example, the health metrics gathered from one or more of the IMUs in a half-second time span may be used as a data segment that corresponds to the first half-second recorded in the video recording. Thus, at least one embodiment described herein may produce dense, consistent, labeled data segments for use in training a model with significantly reduced human labor. Other health metrics such as temperature, skin moisture level, heartbeat, etc. may also be used.

In at least one embodiment, the labeled data segments may be partitioned into training, validation, and test datasets according to predetermined ratios. By way of example and not limitation, the ratios may be, in order of training/validation/test: 70/15/15, 80/10/10, 60/20/20, or 50/30/20. The one or more machine-learning models may be iteratively trained on the training dataset while monitoring validation loss to reduce overfitting.

Model performance may be evaluated using one or more metrics including classification accuracy, precision, recall, F1-score, confusion matrices, receiver operating characteristic (ROC) curves, and/or area-under-curve (AUC) measurements.

In at least one embodiment, cross-validation techniques such as k-fold cross-validation may be employed to improve generalization across different animals, breeds, or environmental conditions.

FIG. 6 shows that the method 600 may include a fifth step 650 of training/generating one or more classification models for interpreting multi-sensor time-series data collected from the wearable device 100. The fifth step 650 may include receiving the labeled, time-aligned multi-sensor data segments generated in the preceding steps and using the labeled segments to train a machine-learning model to classify one or more activities, behaviors, postures, or physiological states of the animal.

In at least one embodiment, the fifth step 650 may include extracting spatial-temporal features from the labeled multi-sensor data, generating feature representations that reflect coordinated motion across multiple anatomical regions, and iteratively updating parameters of the classification model using supervised and/or unsupervised learning techniques to minimize prediction error relative to the assigned labels. The method 600 may further include partitioning the labeled data segments into training, validation, and/or test datasets; adjusting model weights through gradient-based optimization; and evaluating performance metrics such as classification accuracy, precision, recall, or loss values.

In at least one embodiment, during the fifth step 650, the health metrics provided by the plurality of multi-point sensors of the wearable device 100 may enable the classifier model to learn spatial-temporal relationships between the movements of different regions of the dog's body. For example, the combined acceleration and rotational data collected from the neck, chest, and back IMUs can reflect subtle distinctions between similar behaviors, such as the dog lowering its head to drink water versus lowering its head to begin scent investigation. These distinctions may arise from specific combinations of angular velocity, body weighting, and micro-movements across the multiple IMU nodes. As such, the trained classifier model can learn and be trained to detect far more detailed representations of canine behavior than what is achievable using conventional single-sensor collar devices.

The fifth step 650 may also include training and/or generating one or more optimized models that are optimized for low-power embedded processing. In at least one embodiment, the one or more optimized models may be machine learning models or other Al models described herein. In at least one embodiment, the one or more machine learning models may be trained with architectural constraints that limit computational complexity, memory usage, and power consumption so that the one or more machine learning models can be executed directly on the one or more processors 120 of the wearable device 100. Such optimization may include reducing model size through parameter pruning, weight quantization, model compression, knowledge distillation, feature selection, and/or selection of lightweight model architectures suitable for low-power processing.

The one or more optimized models may be configured to perform real-time inference on multi-sensor time-series data while minimizing latency and battery consumption. In this manner, behavior classification and health-state inference may be performed locally on the wearable device 100 without requiring continuous transmission of raw sensor data to an external computing device, which may reduce communication bandwidth requirements and improve energy efficiency.

In at least one embodiment, the one or more optimized models may be optimized for embedded deployment using parameter pruning, weight quantization (such as 8-bit or 16-bit quantization), knowledge distillation, or model compression techniques to reduce memory footprint and computational load.

Additionally or alternatively, the one or more optimized models may be configured to execute inference within a latency threshold and within a predefined power budget suitable for battery-powered wearable operation. In at least one embodiment, the latency threshold may be less than 100 milliseconds per inference window. In at least one embodiment, inference may be performed locally on the wearable device 100, while periodic retraining or model refinement may be performed on the external computing device 240 or a cloud-based system.

In at least one additional or alternative embodiment, the method 600 may include incorporating environmental and/or contextual sensor data to further enrich the feature space and the training of the classifier model(s). For example, audio cues, temperature variations, pressure distributions, and GPS-based motion traces may all contribute to distinguishing between ambiguous states. For example, although the IMU profile of a dog abruptly halting may resemble the beginning of a scent alert, the addition of audio signatures, skin-moisture changes, and/or localized chest-pressure variations may isolate the true behavioral cause. By aligning these multimodal signals with the annotated video frames, the method 600 may provide for the generation of highly discriminative models capable of identifying fine-grained behaviors across varying environments, breeds, and temperaments.

The method 600 is not limited to embodiments that include a wearable device according to at least one embodiment described herein. For example, one skilled in the art will appreciate that the method 600 may be implemented for any time-series data according to at least one embodiment described herein.

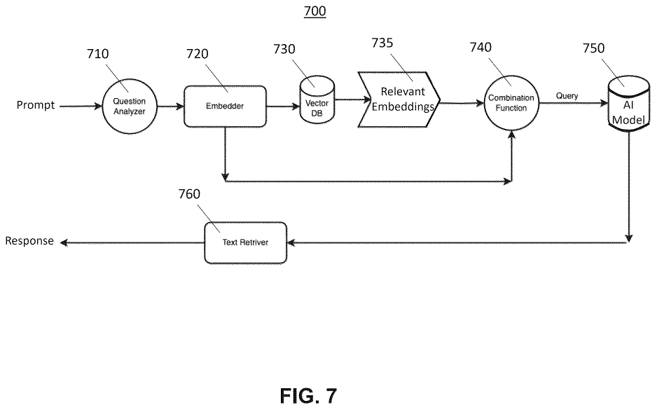

FIG. 7 illustrates a system for smart monitoring of an animal according to at least one embodiment described herein. The system 700 may include a question analyzer 710, an embedder 720, a vector database 730, a combination function 740, an AI model 750, and a text retriever 760. The system 700 may be implemented at least partially by the wearable device 100 and/or the external device 240. In at least one embodiment, lighter weight preprocessing and/or postprocessing may execute on the wearable device 100, while more computationally intensive components such as embedding generation or large language model inference may execute on the external device 240.

As shown in FIG. 7, the system 700 may receive a prompt from a user. The prompt may include user-generated text, transcribed speech input, one or more labeled data segments, and/or one or more current health metrics gathered by the wearable device 100. In at least one embodiment, the prompt may also implicitly include contextual metadata such as time of day, geographic location, recent activity classifications, personality parameters, and/or health-template matching results.

The question analyzer 710 may receive the prompt and perform one or more preprocessing operations. Such preprocessing may include natural language parsing, intent detection, entity extraction, sentiment analysis, classification of question type and/or normalization of sensor-derived inputs. In at least one embodiment, the question analyzer 710 may determine whether the prompt requires retrieval of historical data, real-time physiological data, disease-template comparisons, personality-profile information, or combinations thereof.

The processed prompt may then be passed to the embedder 720. The embedder 720 may include one or more machine-learning models configured to convert textual input, sensor-derived feature vectors, and/or behavioral descriptors into numerical embedding representations within a high-dimensional vector space. In at least one embodiment, different embedding models may be used for textual inputs and sensor-derived inputs, and the resulting embeddings may be projected into a shared embedding space to enable cross-modal and/or multi-modal retrieval.

The embedding generated by the embedder 720 may be stored in the vector database 730. The vector database 730 may include previously stored embeddings corresponding to historical conversations, health metric summaries, personality traits, disease-template matches, activity classifications, training performance logs, veterinary notes, and/or other structured or unstructured data associated with the animal. The vector database 730 may support search operations, such as nearest-neighbor retrieval, cosine similarity ranking, or other distance-based comparison techniques.

FIG. 7 also shows that upon receiving the new embedding, the system 700 may retrieve one or more relevant embeddings 735 from the vector database 730 that satisfy a similarity threshold relative to the new embedding. In at least one embodiment, the relevant embeddings 735 may include relevant historic health metrics, text prompts, or other relevant embeddings. These retrieved embeddings may represent contextually related prior interactions, relevant health conditions, similar behavioral events, or historically comparable physiological patterns.

The combination function 740 may then combine the newly generated embedding with the retrieved embeddings to construct an augmented query. In at least one embodiment, the combination function 740 may concatenate textual summaries corresponding to the retrieved embeddings, weight retrieved results according to similarity scores, filter irrelevant historical data, and/or inject structured data into a formatted prompt template to form an augmented query. In at least one embodiment, the structured data may include current heart rate, activity classification, disease-risk score, or other structured data. The augmented query may therefore include both the user's current prompt and contextually relevant historical and physiological information.

Additionally or alternatively, in at least one embodiment, embeddings stored in the vector database 730 may be indexed using approximate nearest neighbor (ANN) search algorithms such as hierarchical navigable small world (HNSW) graphs, product quantization (PQ), locality-sensitive hashing (LSH), or related indexing methods. Retrieval of relevant embeddings 735 may be performed by computing cosine similarity or dot-product similarity between a query embedding and stored embeddings and selecting the top-K most similar entries.

In at least one additional or alternative embodiment, the combination function 740 may construct an augmented prompt by concatenating retrieved contextual summaries, structured physiological metrics, and personality parameters into a formatted prompt template prior to submission to the AI model 750.

The augmented query may then be passed to the AI model 750. The AI model 750 may include a large language model, transformer-based architecture, RAG model, or other generative model configured to produce natural language responses conditioned on the augmented query. In at least one embodiment, the AI model 750 may further condition response generation on the animal's stored personality profile, current mood inference, and/or health-template matching results to produce a response that reflects both factual health information and a personality-consistent communication style. The AI model 750 may be similar to or the same as the AI model 310.

The text retriever 760 may receive the output of the AI model 750 and provide the response to the user via text display, synthesized speech output, and/or transmission to the wearable device 100 for audio rendering. In at least one embodiment, the text retriever 760 may additionally perform safety filtering, response validation, summarization, or formatting before delivery to the user.

At least one embodiment described herein may thus enable context-aware, physiologically grounded conversational interaction in which responses are generated based not only on a user's question, but also on real-time and historical health metrics, behavior patterns, and learned personality traits of the animal. Thus, at least one embodiment described herein may enable an owner to have early detection of adverse conditions faced by the animal such as an illness or an injury.

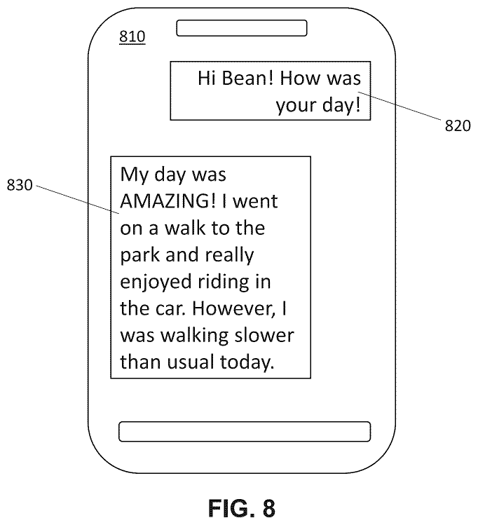

FIG. 8 shows one example of an interaction between an owner and the wearable device according to at least one embodiment described herein. FIG. 8 shows that the owner may interact with the wearable device 100 using a smartphone 810. In at least one embodiment, the owner may send, using the smartphone 810, a text prompt 820 to the wearable device 100. The wearable device 100 may receive the text prompt 820 and then generate a response 830 using the text prompt 820 and at least some of the health metrics gathered by the one or more sensors 110 as described herein. In at least such a way, the owner may be appraised of adverse conditions faced by the animal that is wearing the wearable device 100.

Further, the methods may be practiced by a computer system including one or more processors and computer-readable media such as computer memory. In particular, the computer memory may be a non-transitory storage medium that is configured to store computer-executable instructions that when executed by one or more processors cause various functions to be performed, such as the acts recited in the embodiments.

Computing system functionality can be enhanced by a computing systems' ability to be interconnected to other computing systems via network connections. Network connections may include, but are not limited to, connections via wired or wireless Ethernet, cellular connections, or even computer to computer connections through serial, parallel, USB, or other connections. The connections allow a computing system to access services at other computing systems and to quickly and efficiently receive application data from other computing systems.

Interconnection of computing systems has facilitated distributed computing systems, such as so-called “cloud” computing systems. In this description, “cloud computing” may be systems or resources for enabling ubiquitous, convenient, on-demand network access to a shared pool of configurable computing resources (e.g., networks, servers, storage, applications, services, etc.) that can be provisioned and released with reduced management effort or service provider interaction. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Cloud and remote based service applications are prevalent. Such applications are hosted on public and private remote systems such as clouds and usually offer a set of web based services for communicating back and forth with clients.

Many computers are intended to be used by direct user interaction with the computer. As such, computers have input hardware and software user interfaces to facilitate user interaction. For example, a modern general purpose computer may include a keyboard, mouse, touchpad, camera, etc. for allowing a user to input data into the computer. In addition, various software user interfaces may be available.

Examples of software user interfaces include graphical user interfaces, text command line based user interface, function key or hot key user interfaces, and the like.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

Additionally, unless such is mutually exclusive, terms such as “and”, “or”, and “and/or” are intended to convey that the referenced elements may be present individually, collectively, or in any combination thereof. For example, a reference to “A and/or B” is intended to encompass A alone, B alone, and both A and B together. Similarly, references to “at least one of” a list of elements are intended to include any single element of the list as well as any combination of two or more elements of the list.

Unless otherwise expressly stated, the terms “comprising,” “including,” “having,” and variations thereof are intended to be open-ended and non-limiting. Additionally, singular forms such as “a,” “an,” and “the” are intended to include plural forms unless the context clearly indicates otherwise. Terms such as “configured to,” “adapted to,” or “operable to” are intended to describe structural or functional capability and are not intended to require actual operation in all embodiments.

It will be appreciated that the various embodiments and features described herein are not necessarily mutually exclusive and may be combined in whole or in part with one another unless expressly stated otherwise or unless such combination would be inoperable. Features described in connection with one embodiment may be incorporated into other embodiments, and elements described in connection with particular implementations may be interchanged or substituted with equivalent elements described elsewhere in the specification. Accordingly, the scope of the present disclosure includes combinations and sub-combinations of the described features and embodiments, even if such combinations are not explicitly illustrated or described in a single embodiment.

If a first embodiment and/or component is described herein as being “similar to” or “the same as” a second embodiment and/or component, it should be understood that the structural features, functional characteristics, operational steps, and/or implementation details described with respect to one embodiment or component may be applicable to the other, unless expressly stated otherwise or unless such application would be inoperable. Accordingly, description provided for one embodiment or component may be incorporated by reference into the description of another embodiment or component, and vice versa, even if not repeated in full. The absence of explicit repetition is not intended to limit the scope of the disclosure or to imply that features are restricted to only the embodiment in which they are first described.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A wearable device for smart monitoring of an animal, the wearable device comprising:
an electronics module comprising one or more sensors connected to one or more processors; and
a wearable article, wherein the wearable article is configured to at least partially incorporate the electronics module;
wherein the one or more sensors are configured to gather one or more health metrics from the animal and wherein the electronics module comprises a non-transitory storage medium storing instructions that are configured to be executable by the one or more processors to cause the one or more processors to:
receive the one or more health metrics;
analyze the one or more health metrics to produce a health-related output indicative of at least one of a physiological condition, behavioral state, or activity of the animal; and
cause the health-related output to be communicated to an external computing device and/or presented via one or more outputs of the wearable device.

2. (canceled)

3. The wearable device of claim 1, wherein the one or more sensors comprise a plurality of inertial measurement units (IMUs) positioned at different anatomical positions of the animal.

4. The wearable device of claim 1, wherein the one or more sensors further comprise at least one of a temperature sensor, heart rate sensor, blood oxygen sensor, pressure sensor, microphone, GPS receiver, or inertial measurement unit (IMU), wherein the IMU includes one or more 3-axis accelerometers, 3-axis gyroscopes and/or 3-axis magnetometers with sensor fusion.

5. The wearable device of claim 1, wherein analyzing the one or more health metrics comprises comparing the one or more health metrics to one or more stored health metric templates.

6. (canceled)

7. The wearable device of claim 1, wherein analyzing the one or more health metrics comprises using at least one trained AI model, wherein the at least one trained AI model is a machine-learning classification model and/or a large language model.

8. The wearable device of claim 7, wherein the machine-learning classification model is trained using labeled multi-sensor time-series data to interpret multi-sensor time-series data collected from the wearable device.

9. The wearable device of claim 1, wherein the one or more outputs comprise at least one of a speaker, light emitting diode (LED), or haptic actuator.

10. The wearable device of claim 9, wherein the instructions are further configured to cause the one or more processors to selectively activate the one or more outputs to deliver a command to the animal.

11. The wearable device of claim 1, wherein the instructions further cause the one or more processors to receive a user prompt and generate a natural language response based at least in part on the one or more health metrics.

12. The wearable device of claim 11, wherein generating the natural language response comprises conditioning the natural language response on a stored personality profile associated with the animal.

13. The wearable device of claim 1, wherein the instructions further cause the one or more processors to determine whether the animal has performed a commanded behavior based on the one or more health metrics.

14. A system for smart monitoring and training of an animal, the system comprising:
a wearable device configured to be worn by the animal, the wearable device comprising one or more sensors and one or more first processors;
an external computing device communicatively coupled to the wearable device and comprising one or more second processors;
a treat dispenser communicatively coupled to the wearable device and/or the external computing device; and
one or more non-transitory computer-readable media storing instructions that, when executed by the one or more first processors and/or the one or more second processors, cause the system to:
deliver a command to the animal;
receive from the one or more sensors one or more health metrics associated with the animal;
determine, based on the one or more health metrics, whether the animal performed a behavior corresponding to the command; and
activate the treat dispenser to dispense a treat to the animal when the system determines that the animal performed the behavior corresponding to the command.

15. The system of claim 14, wherein the one or more sensors comprise a plurality of inertial measurement units (IMUs) disposed at different anatomical positions of the animal, and wherein determining whether the animal performed the behavior corresponding to the command comprises:
fusing a plurality of measurements received from the plurality of IMUs to produce a higher-fidelity representation of a posture, gait, and/or fine-grained behavior of the animal, and
determining whether the higher-fidelity representation indicates that the animal performed the behavior corresponding to the command.

16. The system of claim 14, wherein the command comprises at least one of an audio signal, a visual signal, or a haptic vibration pattern delivered by the wearable device.

17. The system of claim 14, wherein the external computing device and/or the wearable device is configured to implement one or more artificial intelligence models that determine, based on the one or more health metrics, whether the animal performed a behavior corresponding to the command.

18. A method for training a machine-learning model to classify animal behavior, the method comprising:
collecting multi-point sensor data from multiple sensors of a wearable device positioned at different anatomical regions of an animal;
generating a timecode signal using the wearable device worn by the animal;
recording video of the animal using a camera which is configured to receive the timecode signal such that each video frame corresponds to a synchronized data segment interval;
analyzing the video to identify one or more activities of the animal;
automatically labeling one or more corresponding synchronized sensor-data intervals with at least one corresponding activity to produce one or more labeled data segments; and
training the machine-learning model using at least some of the one or more labeled data segments.

19. The method of claim 18, wherein analyzing the video comprises using at least one pretrained AI model to analyze the video to identify the one or more activities of the animal.

20. The method of claim 18, further comprising partitioning the one or more labeled data segments into a training dataset and a validation dataset and iteratively updating one or more parameters of the machine-learning model based on the training dataset while evaluating performance of the machine-learning model using the validation dataset.

21. The wearable device of claim 3, wherein there is at least one IMU disposed at a neck of the animal, at least one IMU disposed at a chest of the animal, and at least one IMU disposed at a dorsal region of the animal, and the one or more health metrics comprise a plurality of measurements from the plurality of IMUs, wherein each measurement relates to a motion of the animal.

22. The wearable device of claim 21, wherein the instructions are further configured to cause the one or more processors to fuse the plurality of measurements to produce a higher-fidelity representation of a posture, gait, and/or fine-grained behavior of the animal.
